(19) United States
(12) Patent Application Publication
(10) Pub. No.: US 2026/0106000 A1
(43) Pub. Date: Apr. 16, 2026

(54) MEDICAL CODING DECISION SUPPORT SYSTEM USING MULTIPLE MACHINE-LEARNED MODELS INCLUDING DEEP LEARNING BASED NATURAL LANGUAGE PROCESSING MODELS

(71) Applicant: Optum, Inc., Minnetonka, MN (US)

(72) Inventors: Wenji Zhang, Redmond, WA (US); Yi Tang, Sammamish, WA (US); Feili Yu, Shoreline, WA (US); Zhao Liu, Seattle, WA (US); Hao Zhou, Mercer Island, WA (US)

(21) Appl. No.: 18/914,566

(22) Filed: Oct. 14, 2024

(51) Int. Cl. G16H 10/60; G16H 15/00; G16H 50/20

(52) U.S. Cl. CPC G16H 10/60; G16H 15/00; G16H 50/20

(57) ABSTRACT

A method includes receiving, a record containing clinical information associated with a patient; applying the record a set of machine-learned models including: a medical entity extraction model that is configured to extract medical entities from the clinical information; a medical ontology mapping model that is configured to associate one or more candidate medical codes with each of the medical entities based on vector embedding similarities and to rank each of the one or more candidate medical codes based on their similarities, respectively, with the associated medical entity; and a validation model that is configured to categorize an association between one or more pairs of the one or more candidate medical codes into one or more confidence ratings, respectively; generating a suggested medical code for each of the medical entities based on the rank of the one or more candidate medical codes associated therewith and the one or more confidence ratings of the one or more candidate medical codes, respectively; and causing, via a graphical user interface (GUI), display of the suggested medical code for each of the medical entities and, for each of the suggested medical codes, a corresponding visual indicator that is representative of the confidence rating that is associated with the respective suggested medical code.

### FIELD

The present disclosure relates generally to health care systems and services and, more particularly, to decision support systems for use in coding clinical records.

### BACKGROUND

Health care service providers record clinical information associated with patients under their care in clinical charts, which are typically stored as electronic health records. A chart or health record for a patient may be lengthy sometimes exceeding a thousand pages in length. Health care providers typically assign the health records to coders that review the records and assign codes to the various procedures and diagnoses contained therein. The coders may make use of tools or support systems to assist them in assigning codes to the various portions of clinical information contained within the records. The codes are used in generating claims for the services provided by the health care providers for submission to payors for reimbursement. Various coding libraries can be used including, for example, International Classification of Diseases, Tenth Revision (ICD-10) codes, Current Procedural Terminology (CPT) codes, and Healthcare Common Procedure Coding System (HCPCS) codes. When reviewing claims for payment, payors may also obtain patient medical records and may review the records to identify the evidence within the records and codes associated therewith to support the various services for which the provider is seeking payment. Due to the length of a chart of health record for a patient, there may be thousands of medical codes that are applicable to any given document. Moreover, the differences between many of the codes may be subtle. As a result, the process of coding a medical record for use in claim generation, for example, may require extensive manual labor and may also be prone to error.

### SUMMARY

According to some embodiments of the disclosure, a computer-implemented method comprises: receiving, by one or more processors, a record containing clinical information associated with a patient; applying, by the one or more processors and to the record, a set of machine-learned models including: a medical entity extraction model that is configured to extract medical entities from the clinical information; a medical ontology mapping model that is configured to associate one or more candidate medical codes with each of the medical entities based on vector embedding similarities and to rank each of the one or more candidate medical codes based on their similarities, respectively, with the associated medical entity; and a validation model that is configured to categorize, an association between one or more pairs of the one or more candidate medical codes into one or more confidence ratings, respectively; generating, by the one or more processors, a suggested medical code for each of the medical entities based on the rank of the one or more candidate medical codes associated therewith and the one or more confidence ratings of the one or more candidate medical codes, respectively; and causing, by the one or more processors and via a graphical user interface (GUI), display of the suggested medical code for each of the medical entities and, for each of the suggested medical codes, a corresponding visual indicator that is representative of the confidence rating that is associated with the respective suggested medical code.

In other embodiments, the medical entity extraction model comprises a bidirectional encoder representations from transformers (BERT) model that is trained using annotated historic patient clinical information records.

In still other embodiments, the method further comprises: generating, using the medical entity extraction model and the one or more processors, a set of candidate medical entities from the clinical information; generating, using the medical entity extraction model and the one or more processors, medical entity confidence scores for the candidate medical entities, respectively, using Gibbs sampling to perform a statistical inference based on the candidate medical entities and a medical entity knowledge base that contains quantified measures of relationship strength between pairs of reference medical entities; and extracting, by the one or more processors, the medical entities from the clinical information based on the confidence scores for the candidate medical entities.

In still other embodiments, the method further comprises: categorizing, using the medical entity extraction model and the one or more processors, each of the extracted medical entities into one of a set of medical entity categories.

In still other embodiments, the set of medical entity categories comprises a disease category, a medication category, a dosage category, a form category, a route category, a procedure category, a lab category, and a symptom category.

In still other embodiments, the method further comprises: generating, using the medical ontology mapping model and the one or more processors, a medical entity vector embedding for each of the medical entities; generating, using the medical ontology mapping model and the one or more processors, a reference code vector embedding for each of a set of reference medical codes; and determining, using the medical ontology mapping model and the one or more processors, the vector embedding similarities between ones of the medical entity vector embeddings and ones of the set of reference medical code vector embeddings.

In still other embodiments, determining the vector embedding similarities comprises: determining, using the medical ontology mapping model and the one or more processors, a set of logits corresponding to a set of inner products between the ones of the medical entity vector embeddings and the ones of the set of reference medical code vector embeddings.

In still other embodiments, determining the vector embedding similarities further comprises: applying, using the medical ontology mapping model and the one or more processors, a sigmoid function to each of the set of logits to generate a set of similarity probabilities.

In still other embodiments, the method further comprises: determining, using the medical ontology mapping model and the one or more processors, whether each of the set of similarity probabilities satisfies a similarity threshold to generate a set of similarity comparison results; associating, using the medical ontology mapping model and the one or more processors, the one or more candidate medical codes with each of the medical entities based on the similarity comparison results; and ranking, using the medical ontology mapping model and the one or more processors, each of the one or more candidate medical codes associated with each of the medical entities based on the similarity probabilities.

In still other embodiments, the method further comprises: generating, using the validation model and the one or more processors, first confidence scores for reference medical code pairs based on conditional probabilities for the pairs, respectively; performing, using the validation model and the one or more processors, mean shift unsupervised clustering on the pairs to generate clusters; determining, using the validation model and the one or more processors, mean conditional probabilities for the clusters; generating, using the validation model and the one or more processors, second confidence scores for the reference medical code pairs based on the first confidence scores and the mean conditional probabilities for the clusters; and categorizing, using the validation model and the one or more processors, the association between one or more pairs of the one or more candidate medical codes into the one or more confidence ratings based on the second confidence scores, respectively.

In still other embodiments, the one or more confidence ratings reflect varying degrees of confidence that respective ones of the one or more pairs of the one or more candidate medical codes are correlated.

In still other embodiments, the suggested medical code is an International Classification of Diseases (ICD) code, a Current Procedural Terminology (CPT) code, or a Healthcare Common Procedure Coding System (HCPCS) code.

In some embodiments of the disclosure, a system comprises one or more processors; and one or more memories storing process-executable instructions that, when executed by the one or more processors, cause the one or more processors to perform operations comprising: receiving a record containing clinical information associated with a patient; applying, to the record, a set of machine-learned models including: a medical entity extraction model that is configured to extract medical entities from the clinical information; a medical ontology mapping model that is configured to associate one or more candidate medical codes with each of the medical entities based on vector embedding similarities and to rank each of the one or more candidate medical codes based on their similarities, respectively, with the associated medical entity; and a validation model that is configured to categorize, an association between one or more pairs of candidate medical codes into one or more confidence ratings, respectively; generating a suggested medical code for each of the medical entities based on the rank of the one or more candidate medical codes associated therewith and the one or more confidence ratings of the one or more candidate medical codes, respectively; and causing, via a graphical user interface (GUI), display of the suggested medical code for each of the medical entities and, for each of the suggested medical codes, a corresponding visual indicator that is representative of the confidence rating that is associated with the respective suggested medical code.

In further embodiments, the medical entity extraction model comprises a bidirectional encoder representations from transformers (BERT) model that is trained using annotated historic patient clinical information records.

In still further embodiments, the operations further comprise: generating, using the medical entity extraction model, a set of candidate medical entities from the clinical information; generating, using the medical entity extraction model, medical entity confidence scores for the candidate medical entities, respectively, using Gibbs sampling to perform a statistical inference based on the candidate medical entities and a medical entity knowledge base that contains quantified measures of relationship strength between pairs of reference medical entities; and extracting the medical entities from the clinical information based on the confidence scores for the candidate medical entities.

In still further embodiments, the operations further comprise: categorizing, using the medical entity extraction model, each of the extracted medical entities into one of a set of medical entity categories.

In still further embodiments, the operations further comprise: generating, using the medical ontology mapping model, a medical entity vector embedding for each of the medical entities; generating, using the medical ontology mapping model, a reference code vector embedding for each of a set of reference medical codes; determining, using the medical ontology mapping model, the vector embedding similarities between ones of the medical entity vector embeddings and ones of the set of reference medical code vector embeddings.

In still further embodiments, the operations further comprise: generating, using the validation model, first confidence scores for reference medical code pairs based on conditional probabilities for the pairs, respectively; performing, using the validation model, mean shift unsupervised clustering on the pairs to generate clusters; determining, using the validation model and, mean conditional probabilities for the clusters; generating, using the validation model, second confidence scores for the reference medical code pairs based on the first confidence scores and the mean conditional probabilities for the clusters; and categorizing using the validation model, the association between and one or more pairs of the one or more candidate medical codes into the one or more confidence ratings based on the second confidence scores, respectively.

In still further embodiments, the one or more confidence ratings reflect varying degrees of confidence that respective ones of the one or more pairs of the one or more candidate medical codes are correlated.

In some embodiments, one or more non-transitory computer-readable media storing processor-executable instructions that, when executed by one or more processors, cause the one or more processors to perform operations comprising: receiving, by the one or more processors, a record containing clinical information associated with a patient; applying, to the record, a set of machine-learned models including: a medical entity extraction model that is configured to extract medical entities from the clinical information; a medical ontology mapping model that is configured to associate one or more candidate medical codes with each of the medical entities based on vector embedding similarities and to rank each of the one or more candidate medical codes based on their similarities, respectively, with the associated medical entity; and a validation model that is configured to categorize an association between one or more pairs of the one or more candidate medical codes into one or more confidence ratings, respectively; generating a suggested medical code for each of the medical entities based on the rank of the one or more candidate medical codes associated therewith and the one or more confidence ratings of the one or more candidate medical codes, respectively; and causing, via a graphical user interface (GUI), display of the suggested medical code for each of the medical entities and, for each of the suggested medical codes, a corresponding visual indicator that is representative of the confidence rating that is associated with the respective suggested medical code.

Other methods, systems, articles of manufacture, and/or computer program products according to embodiments of the disclosure will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including an intelligent coding Decision Support System (DSS) in accordance with some embodiments of the disclosure;

FIG. 2 is a block diagram of the intelligent coding DSS in accordance with some embodiments of the disclosure;

FIG. 3 is a block diagram of a medical entity extraction system in accordance with some embodiments of the disclosure;

FIG. 4 is a block diagram of a medical ontology mapping system in accordance with some embodiments of the disclosure;

FIG. 5 is a block diagram of a validation system in accordance with some embodiments of the disclosure;

FIGS. 6 and 7 are flowcharts that illustrate operations of the intelligent coding DSS in accordance with some embodiments of the disclosure;

FIG. 8 is a graph that illustrates generation of an inner product between two vectors;

FIGS. 9 and 10 are flowcharts that illustrate operations of the intelligent coding DSS in accordance with further embodiments of the disclosure;

FIG. 11 is a data processing system that may be used to implement an intelligent coding DSS in accordance with some embodiments of the disclosure; and

FIG. 12 is a block diagram that illustrates a software/hardware architecture for use in an intelligent coding DSS in accordance with some embodiments of the disclosure.

### DETAILED DESCRIPTION

In the following detailed description, numerous specific details are set forth to provide a thorough understanding of embodiments of the disclosure. However, it will be understood by those skilled in the art that embodiments of the disclosure may be practiced without these specific details. In some instances, well-known methods, procedures, components, and circuits have not been described in detail so as not to obscure embodiments of the disclosure. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

As used herein a “procedure” may be, but is not limited to, any type of treatment provided by a provider to a patient or any type of medicine or product prescribed or given to a patient for treatment. In general, a “procedure” may be defined as any activity directed at or performed on an individual with the object of improving health, treating disease or injury, or making a diagnosis.

Embodiments of the disclosure are described herein in the context of a Decision Support System (DSS) that includes one or more Artificial Intelligence (AI) models or machine-learned models for processing patient records, which include clinical information, and suggesting codes therefor. The one or more AI models of the intelligent coding DSS be embodied in a variety of different ways including, but not limited to, one or more of the following AI systems: a multi-layer neural network, a machine learning system, a deep learning system, a large language model, a natural language processing system, and/or computer vision system. Moreover, it will be understood that the multi-layer neural network is a multi-layer artificial neural network comprising artificial neurons or nodes and does not include a biological neural network comprising real biological neurons. The AI models described herein may be configured to transform a memory of a computer system to include one or more data structures, such as, but not limited to, arrays, extensible arrays, linked lists, binary trees, balanced trees, heaps, stacks, and/or queues. These data structures can be configured or modified through the adjudication process and/or the AI training process to improve the efficiency of a computer system when the computer system operates in an inference mode to make an inference, prediction, classification, suggestion, or the like with respect to associating medical codes with clinical information.

Embodiments of the disclosure are described herein with respect to use of a DSS for processing clinical patient records and suggesting codes therefor. These codes may include, but are not limited to, International Classification of Diseases, Tenth Revision (ICD-10) codes, Current Procedural Terminology (CPT) codes, and/or Healthcare Common Procedure Coding System (HCPCS) codes. It will be understood that embodiments of the disclosure are not limited to use of a DSS for processing clinical patient records and suggesting codes therefor, but can be applied to classification problems in general

A typical workflow for coding clinical medical records associated with patients involves a mixture of automation, third-party tools, and manual effort. For example, in the example of a payor reviewing medical records to search for evidence and/or codes from the record that would support one or more line items on a claim, the process may begin by filtering medical records using an internal tool to identify the more high value records. Human agents may then split the records manually into multiple files with each file representing an encounter with a provider. A third-party tool may then be used in suggesting codes with the agents approving or rejecting the codes based on evidence highlighted by the tool in the record or human labelers may label spans of text with named entity recognition labels associated with defined medical codes. This workflow, however, may suffer from various drawbacks including, for example, low productivity due to the mixture of manual and automated processes, which may be more complex as both internal and third-party tools may be used. The workflow may have relatively high costs associated therewith due to the intensive human agent review that is involved and the use of third-party vendors to supply tools. The workflow may also be subject to market risk due the dependency on vendors for tools. If a vendor does not continue to support a tool after a contract expires, for example, the entire workflow may be impacted. Training may be performed on only a relatively small subset of data and coding may be limited to a subset of medical codes that the labelers have labeled frequently. Research efforts have been made in automating the coding task in both machine learning approaches and rule-based dictionary approaches. These techniques often suffer from a lack of complex reasoning capabilities and insufficient contextual understanding abilities. Another area of research is the use of medical knowledge databases to assist in medical coding. These data sources, however, usually lack clinical context and information on the inter-relationships between medical entities, such as, for example, problems, diagnoses, complaints, symptoms, encounters, allergies, medications, vaccinations, assessments, diagnostic procedures, treatments/therapies, referrals, consults, and/or care plans.

Some embodiments of the disclosure stem from a recognition of the above-described costs, risks, and deficiencies in extracting codes from a medical record in which a combination of human effort, internal support tools, and third-party support tools may be used. Some embodiments of the disclosure may provide an intelligent coding DSS including that makes use of multiple types of AI models including a deep learning model, a Natural Language Processing (NLP) model, and a statistical knowledge model to combine their outputs to generate suggested codes for medical entities found in a record containing clinical information. The AI models may be supplemented with a medical knowledge base to verify that the extracted medical entities from the clinical information are relevant to the coding process.

According to some embodiments of the disclosure, a record containing clinical information associated with a patient may be processed using a plurality of models including: a medical entity extraction model, which may be a deep learning based Named Entity Recognition (NER) model that is configured to extract medical entities from the clinical information; a medical ontology mapping model, which may be an NLP based model that is configured to associate one or more candidate medical codes with each of the medical entities based on vector embedding similarities and is further configured to rank each of the one or more candidate medical codes based on their similarities, respectively, with the associated medical entity; and a validation model, which may be a statistical knowledge-based model that is configured to categorize the association between the one or more candidate medical codes into one or more confidence ratings, respectively. The medical entity extraction model and the medical ontology model are based on NLP and are used to extract and/or highlight medical entities from the clinical information and then identify candidate medical codes for the different entities. Applying NER to medical or clinical documents can result in poor highlighting or extraction results due to the non-standard usage of terms, abbreviations, synonyms, acronyms, and ambiguity in entity descriptions. Advantageously, the medical entity extraction model according to the present disclosure may be implemented as a deep learning NER model where self-annotated medical texts are used as training data to improve the accuracy recognizing medical entities in the clinical information. The AI validation model may provide a supplemental evaluation of the candidate codes output for the different medical entities from the medical entity extraction model and the medical ontology mapping model. The various candidate codes may be categorized or labeled with a confidence rating that reflects the degree of confidence that that different candidate codes are related. Advantageously, the AI validation model may make use of a medical entity knowledge base with historical medical claim data to develop confidence ratings between pairs of medical entities and codes. In some embodiments, a mean shift clustering algorithm may be used to identify conditional probabilities between candidate codes that are associated with an entity. Thus, embodiments of the disclosure may combine the outputs of multiple types of AI models to generate candidate codes for medical entities found in clinical information and may make use of a medical knowledge base to develop confidence ratings between pairings of codes with medical entities to, advantageously, act as a check on the suggested code output from the AI models by identifying those coding suggestions that may warrant special attention. The suggested code that is recommended from the candidate codes for each of the medical entities based on the candidate code rankings and the confidence ratings is displayed via a graphical user interface (GUI) display along with a corresponding visual indicator that is representative of the confidence rating that is associated with the suggested medical code. For example, the GUI could display a medical entity, a suggested medical code therefor, and a confidence rating for the suggested medical code in proximity to one another so that it is clear to a user that all three items are associated with one another. Advantageously, such a display provides an improvement over conventional GUIs as a user is able to view both the suggested medical code and the confidence rating associated with the suggested medical code alongside or proximate to the medical entity allowing the user to make a rapid assessment of the applicability of the suggested or recommended medical code for the particular medical entity.

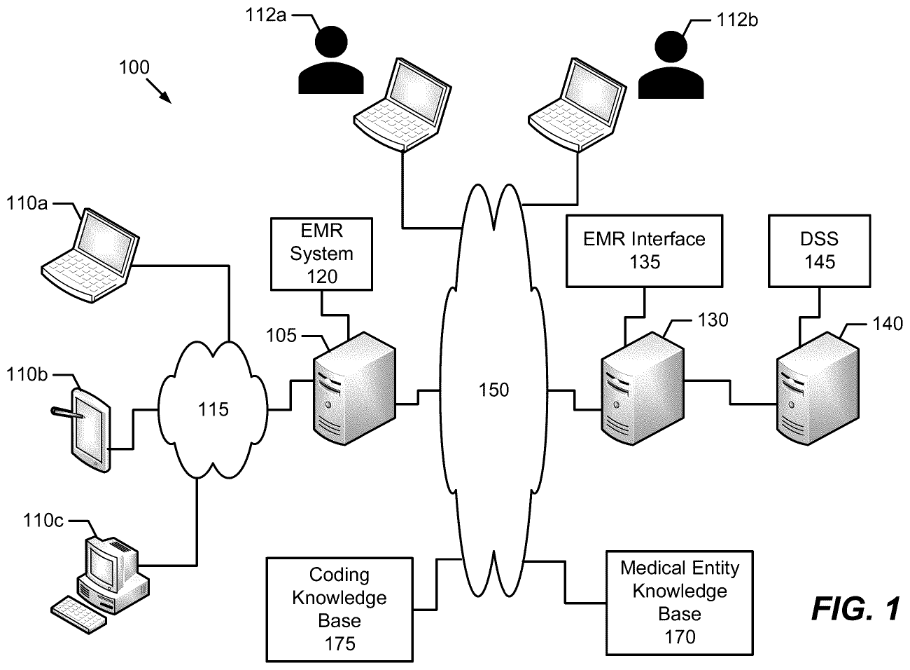

Referring to FIG. 1, a communication network 100 including an intelligent coding DSS, in accordance with some embodiments of the disclosure, comprises a health care facility server 105 that is coupled to devices 110a, 110b, and 110c via a network 115. The health care facility may be any type of health care or medical facility, such as a hospital, doctor's office, specialty center (e.g., surgical center, orthopedic center, laboratory center etc.), or the like. The health care facility server 105 may be configured with an Electronic Medical Record (EMR) system module 120 to manage patient files and facilitate the entry of orders for patients via health care service providers (“providers”). Although shown as one combined system in FIG. 1, it will be understood that some health care facilities use separate systems for electronic medical record management and order entry management. The providers may use devices, such as devices 110a, 110b, and 110c to manage patients'electronic charts or records and to issue orders for the patients through the EMR system 120. An order may include, but is not limited to, a treatment, a procedure (e.g., surgical procedure, physical therapy procedure, radiologic/imaging procedure, etc.) a test, a prescription, and the like. The network 115 communicatively couples the devices 110a, 110b, and 110c to the health care facility server 105. The network 115 may comprise one or more local or wireless networks to communicate with the health care facility server 105 when the health care facility server 105 is located in or proximate to the health care facility. When the health care facility server 105 is in a remote location from the health care facility, such as part of a cloud computing system or at a central computing center, then the network 115 may include one or more wide area or global networks, such as the Internet.

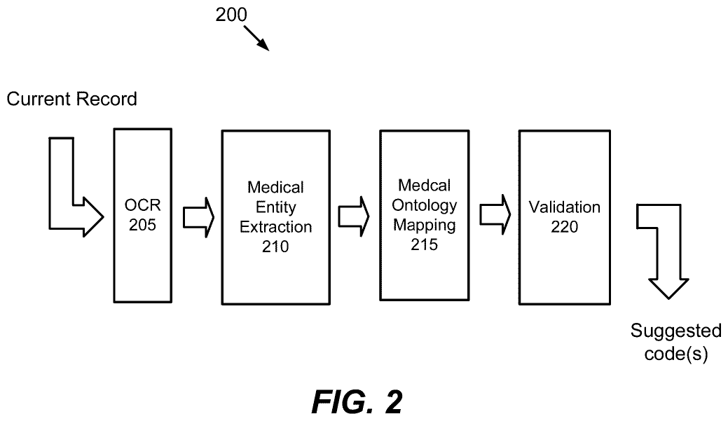

According to some embodiments of the disclosure, an intelligent coding DSS may be provided to assist entities, such as providers, payors, auditors, data entry personnel, and others, which are represented as users 112a and 112b in FIG. 1, in coding patient records, extracting evidence for codes from patient records, auditing existing codes and clinical information corresponding thereto, and the like. The intelligent coding DSS may include a health care facility interface server 130, which includes an EMR interface system module 135 to facilitate the transfer of information between the EMR system 120, which the providers use to manage patient charts and records and issue orders, and a coding suggestion server 140, which includes a DSS module 145. The coding suggestion server 140 and DSS module 145 may be configured to receive patient records from the EMR system 120 by way of the health care facility interface server 130 and EMR interface module 135. The coding suggestion server 140 and DSS module 145 may process each page of each patient clinical record using an AI supported DSS as will be described below with respect to FIG. 2 to generate coding suggestions for one or more portions of the clinical information contained therein. The AI supported DSS may make use of information repositories, such as a medical entity knowledge base 170 and a coding knowledge base 175 as described below.

It will be understood that the division of functionality described herein between the coding suggestion server 140/DSS module 145 and the health care facility interface server 130/EMR interface module 135 is an example. Various functionality and capabilities can be moved between the coding suggestion server 140/DSS module 145 and the health care facility interface server 130/EMR interface module 135 in accordance with different embodiments of the disclosure. Moreover, in some embodiments, the coding suggestion server 140/DSS module 145 and the health care facility interface server 130/EMR interface module 135 may be merged as a single logical and/or physical entity.

A network 150 couples the health care facility server 105, the health care facility interface server 130, and the users 112a, 112b together. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The coding suggestion service provided through the health care facility interface server 130, EMR interface module 135, coding suggestion server 140 and DSS module 145 to automatically suggest codes for one or more portions of clinical information in a patient clinical record may, in some embodiments, be embodied as a cloud service. For example, entities may integrate their clinical record processing system with the code suggestion service and access the service as a Web service. In some embodiments, the code suggestion service may be implemented as a Representational State Transfer Web Service (RESTful Web service).

Although FIG. 1 illustrates an example communication network including an intelligent coding DSS for suggesting codes for one or more portions of a patient clinical record, it will be understood that embodiments of the inventive subject matter are not limited to such configurations, but are intended to encompass any configuration capable of carrying out the operations described herein.

FIG. 2 is a block diagram illustrating a multi-stage AI supported DSS 200 used in the coding suggestion server 140 and DSS module 145 of FIG. 1 in accordance with some embodiments of the disclosure. As shown in FIG. 2, the multi-stage AI supported DSS 200 includes a plurality of modules coupled in pipeline fashion. The multi-stage AI supported DSS 200 may be configured automate the operations involved in generating suggested codes based on a patient's clinical record and then presenting the suggested codes to a user for acceptance or rejection. The multi-stage AI supported DSS 200 includes the following serially connected modules: an Optical Character Recognition (OCR) module 205 configured to convert the patient record into a text record; a medical entity extraction model 210, which may embodied as a deep learning based NER model, that is configured to extract medical entities from clinical information included in one or more patient health records; a medical ontology mapping model 215, which is a NLP based model that is configured to associate one or more candidate medical codes with each of the medical entities based on vector embedding similarities and is further configured to rank each of these candidate medical codes based on their similarities with the associated medical entity; and a validation model 220, which is a statistical knowledge-based model that is configured to categorize the association between the one or more candidate medical codes into one or more confidence ratings. The output of the validation model is one or more suggested medical codes for each of the medical entities extracted from the clinical information in the health care record. The suggested medical codes may be based on the rankings provided by the medical ontology mapping model 215 and the confidence ratings provided by the validation model 220.

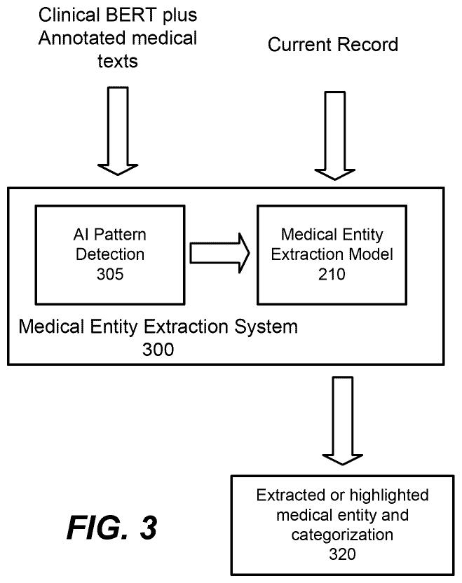

FIG. 3 is a block diagram of a medical entity extraction system 300 that may be used to provide the medical entity extraction model 210 of FIG. 2 in accordance with some embodiments of the disclosure. The medical extraction system 300 may be configured to generate a medical entity extraction model, which may be embodied as an NER model. NER is a form of NLP that involves extracting and identifying essential information from text. The information that is extracted and categorized is called an entity. It can be any word or a series of words that consistently refers to the same thing. According to some embodiments of the disclosure, the medical entity extraction system 300 is configured to classify named entities into the following pre-defined categories: disease (e.g., medical conditions and diagnoses), medication (e.g., pharmaceutical drugs or drug therapies used for treatment), procedure (e.g., diagnostic procedures, therapeutic procedures, and medical devices), lab (e.g., pathology and laboratory procedures), and symptom (e.g., clinical signs and symptoms). Within the medication category, the following sub-categories may be used: dosage (e.g., amount or strength of a drug including units), form (e.g., the physical form of a dose of drug when it is administered), and route (e.g., the way in which a drug is taken into the body). Different types of coding systems may map to different types of medical entity categories. The categories used classify the medical entities may span the different types of coding systems that are used to code the medical entities. The medical entity extraction system 300 includes an AI pattern detection module 305 and the medical entity extraction model 210. The AI pattern detection module 305 may be configured to receive, for example, a machine learning model, such as ClinicalBERT. BERT is a deep neural network that uses the transformer encoder architecture to learn embeddings for text. The transformer encoder architecture is based on a self-attention mechanism. ClinicalBERT is publicly available application of the BERT model to clinical information. The AI pattern detection module 305 may further train the ClinicalBERT model with annotated medical texts to generate the medical entity extraction model 210. During training, the AI pattern detection module 305 learns associations between names of objects in the clinical text and relevant medical entities. Due to the non-standard usage of terms, abbreviations, synonyms, acronyms, and ambiguity in entity descriptions, a supervised deep learning based NER model is used to perform the medical entity extraction to improve the accuracy in identifying medical entities in clinical information, such as patient health records. The medical entity extraction model 210 may be configured to extract or highlight medical entities 320 contained in clinical information of a current record.

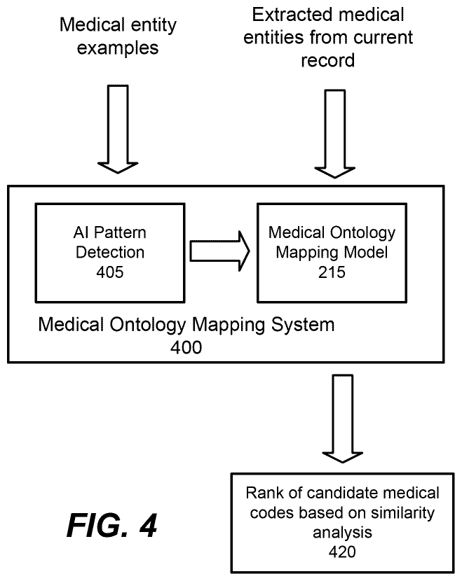

FIG. 4 is a block diagram of a medical ontology mapping system 400 that may be used to provide the medical ontology mapping model 215 of FIG. 2 in accordance with some embodiments of the disclosure. The medical ontology mapping system 400 may include an AI pattern detection module 405 and the medical ontology mapping model 215. The AI pattern detection module 405 may be configured to receive medical entity examples and is trained to convert these examples into biomedical embeddings, e.g., vector embeddings that represent the different medical entities. The AI pattern detection module 405 may generate the medical ontology mapping model 215 based on the learned associations for converting medical entities into biomedical embeddings. The medical ontology mapping model 215 may be configured to receive extracted medical entities from a current record, generate vector embeddings for each of the extracted medical entities, generate vector embeddings for reference medical codes, and determine vector embedding similarities between the vector representations of the extracted medical entities and the vector representations of the reference medical codes. Based on a similarity analysis, a set of candidate medical codes 420 is output for each of the extracted medical entities. The reference medical codes may vary based on the NER classification output from the medical entity extraction model. For example, disease classified medical entities may map to ICD-10 codes, procedure and lab classified medical entities may map to CPT codes, and medication medical entities may map to HCPCS codes.

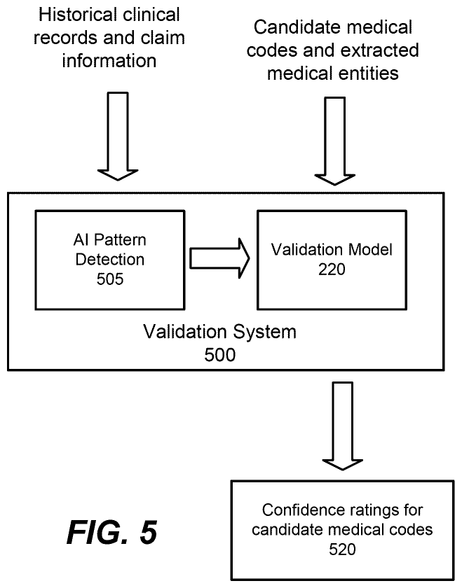

FIG. 5 is a block diagram of a validation system 500 that may be used to provide the validation model 220 of FIG. 2 in accordance with some embodiments of the disclosure. The medical ontology mapping system 500 may include an AI pattern detection module 505 and the validation model 220. The AI pattern detection module 505 may be configured to receive historical clinical records and claim information included coded medical entities from the coding knowledge base 175 of FIG. 1 and is trained to learn statistical associations between the medical entities and codes. The AI pattern detection module 505 generates the validation model 220 based on these learned statistical associations. The validation model 220 may be configured to receive the candidate medical codes associated with each of the extracted medical entities that are output from the medical ontology model 215 and may assign confidence ratings to pairings of the candidate medical codes. The confidence rating may reflect the degree of confidence that two candidate medical codes are correlated. For example, three confidence ratings may be used: high confidence, review needed, and red-flag, which indicate strong, moderate, and low correlations between a medical entity and associated code. In some embodiments, the confidence ratings may be based on conditional probabilities that two candidate medical codes are associated with one through use of a mean shift clustering algorithm.

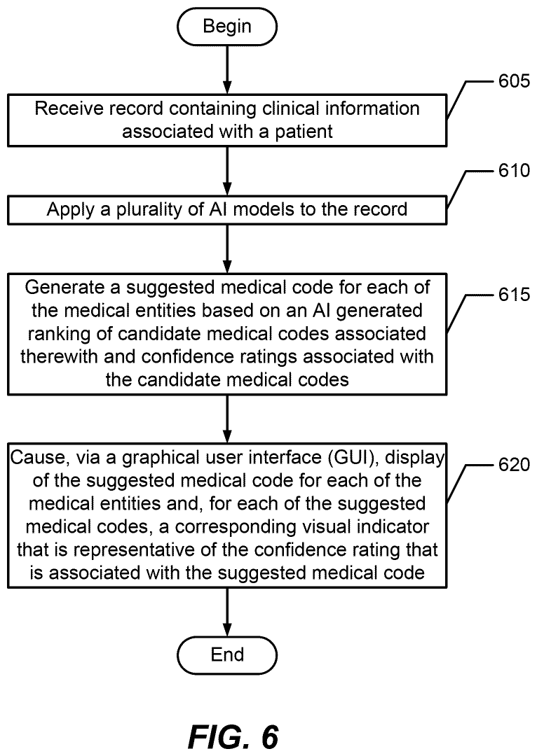

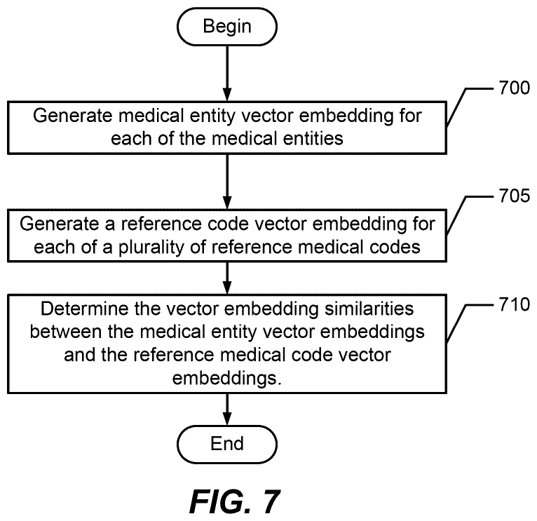

FIGS. 6 and 7 are flowcharts that illustrate operations of the intelligent coding DSS 200 in accordance with some embodiments of the disclosure. Referring now to FIG. 6, operations begin at block 605 where a record containing clinical information associated with a patient is received. A plurality of AI models are applied to the record, such as the medical entity extraction model 210, the medical ontology mapping model 215, and the validation model 220 of FIG. 2 at block 610. In some instances, not all of the medical entities extracted from the clinical information may be relevant. For example, a past medical history section for a patient may include historical medial diagnoses that are no longer current. Coding personnel may examine evidence from other sections of a medical record, such as administered medications, to determine whether a particular disease is current. According to some embodiments of the disclosure the medical entity knowledge base 170 may be used, which identifies relationships between medical entities, e.g., diagnosis-drug, diagnosis-procedures, procedures-drugs, etc., and quantifies the strength of the relationship based on the frequency of the pairing in the medical entity knowledge base. Based on these quantified relationship strength values, a sampling algorithm may be used to generate confidence scores for each of the candidate medical entities extracted using the medical entity extraction model 210. In some embodiments, the Gibbs sampling algorithm may be used to perform the statistical inference. Thus, a set of medical entities may be obtained from the candidate medical entities that are likely to be currently relevant to the patient's care. A suggested medical code for each of the medical entities extracted from the clinical information contained in the record is generated at block 615 based on a ranking of candidate medical codes and confidence ratings associated with the candidate medical codes. The suggested code that is recommended from the candidate codes for each of the medical entities based on the candidate code rankings and the confidence ratings is displayed via a graphical user interface (GUI) display at block 620. In addition to the suggested code, a corresponding visual indicator is also displayed that is representative of the confidence rating that is associated with the suggested medical code. In some embodiments, the GUI could display a medical entity, a suggested medical code therefor, and a confidence rating for the suggested medical code in proximity to one another so that it is clear to a user that all three items are associated with one another.

As described above, the medical ontology mapping model 215 may be configured to receive extracted medical entities from a current record, generate vector embeddings for each of the extracted medical entities, generate vector embeddings for reference medical codes, and determine vector embedding similarities between the vector representations of the extracted medical entities and the vector representations of the reference medical codes. Referring to FIG. 7, example operations of the medical ontology mapping model 215 begin at block 700 where a medical entity vector embedding is generated for each of the extracted medical entities. A reference code vector embedding is generated for each of a plurality of reference medical codes at block 705. In some embodiments, the reference medical codes may be one or more codes from various types of coding standards including, but not limited to, ICD-10 codes, CPT codes, and/or HCPCS codes. Vector embedding similarities between the medical entity vector embeddings and the reference medical code vector embeddings are determined at block 710.

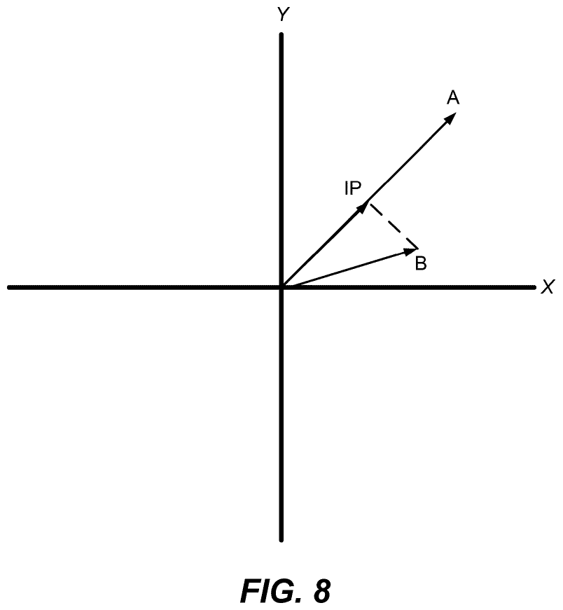

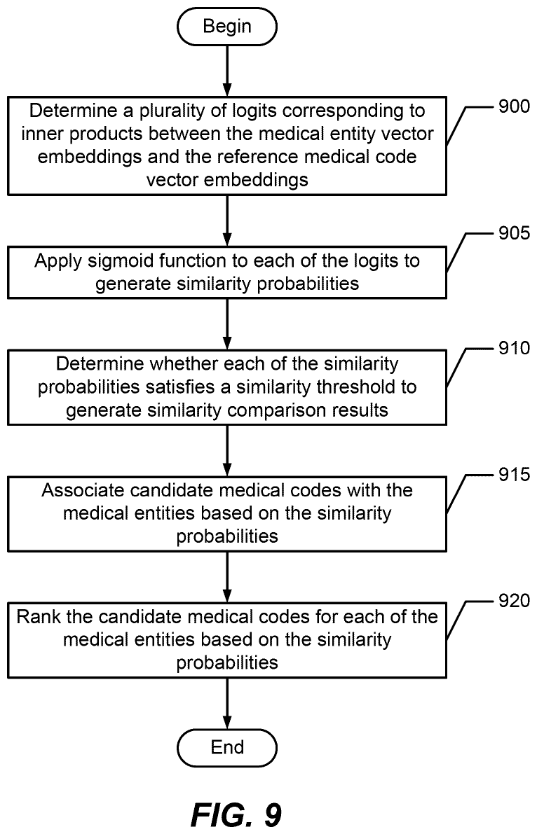

Referring to FIGS. 8 and 9 the similarities between the medical entity vector embeddings and the reference medical code vector embeddings can be determined in a variety of ways. One technique for determining the similarity is to determine logits that correspond to the inner products (e.g., dot products) between the medical entity vector embeddings and the reference medical code vector embeddings at block 900. The inner product between two two-dimensional vectors is illustrated, for example, in FIG. 8. As shown in FIG. 8 the inner product IP between two vectors A and B may be represented as a projection of the vector B onto the vector A. Note that the inner product could be negative depending on the orientation of the two vectors. Returning to FIG. 9, a sigmoid function may be applied to each of the plurality of logits at block 905 to generate normalize the logits and generate a plurality of similarity probabilities. Each of the similarity probabilities may be compared to a threshold to determine whether the similarity satisfies the threshold to generate similarity comparison results at block 910. Candidate medical codes may be associated with the extracted medical entities based on the similarity probabilities at block 915. The candidate medical codes for each of the medical entities can be ranked based on the similarity probabilities to obtain the top k candidate medical codes for each of the medical entities. The number of codes k that are used in the ranking can be selected based on the number of codes associated with a medical entity. While the dot product approach for determining similarity between the medical entity vector embeddings and the reference medical code vector embeddings is one example for determining similarities, other examples include, but are not limited to, using Euclidean distance, Manhattan distance, Chebyshev distance, and/or cosine distance between the vectors.

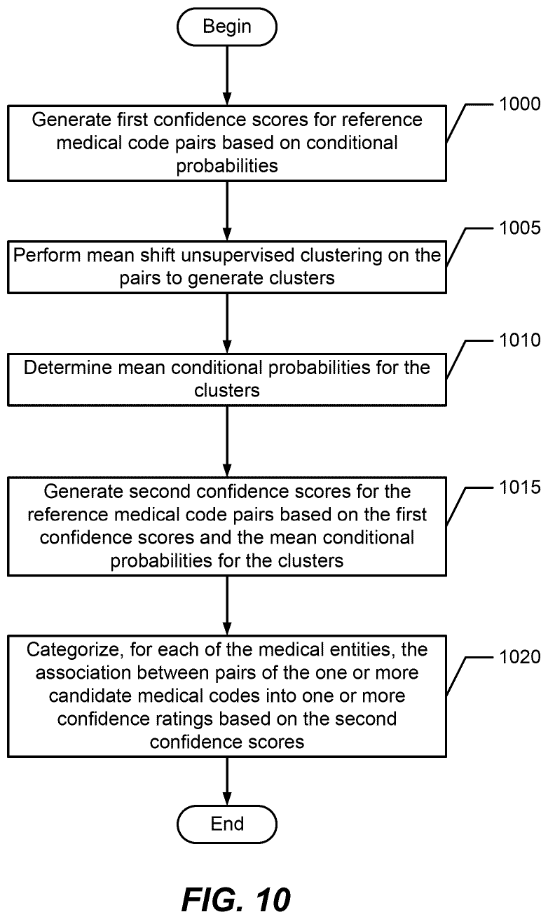

As described above, the validation model 220 may be configured to receive the candidate medical codes associated with each of the extracted medical entities that are output from the medical ontology model 215 and may assign confidence ratings to pairings of candidate codes. The confidence rating may reflect the degree of confidence that two candidate codes are correlated. In some embodiments, the confidence ratings may be based on conditional probabilities that two candidate medical codes are associated with one through use of a mean shift clustering algorithm. Referring now to FIG. 10, operations of the validation model 220 begin at block 1000 where first confidence scores for reference medical code pairs from the coding knowledge base 175 are generated. These confidence scores are based on their conditional probabilities. Mean Shift unsupervised clustering is performed on the pairs to generate clusters at block 1005. Mean Shift is an unsupervised clustering algorithm that is used to discover blobs in a density of samples. Mean Shift is a centroid-based algorithm that works by updating candidates for centroids to be the mean of the points within a given region, which is also called bandwidth. Mean conditional probabilities are determined for the clusters at block 1010. Second confidence scores for the reference medical code pairs are generated at block 1015 based on the first confidence scores and the mean conditional probabilities for the clusters. For each of the medical entities, the association between pairs of the one or more candidate medical codes are categorized into one or more confidence ratings based on the second confidence scores. As described above, in some embodiments, three confidence ratings may be used: high confidence, review needed, and red-flag, which indicate strong, moderate, and low correlations between a medical entity and associated code. For example, the output of the medical ontology mapping model 215 may be a medical entity with a highly ranked candidate code. Based on the historical information contained in the medical entity knowledge base 170 and extracted using the validation model 220, it may be determined that the highly ranked candidate code has historically had a moderate correlation with another candidate code resulting in a “review needed” categorization from the validation model 220. This may result in a coder more closely scrutinizing the assignment of the suggested code based on the ranking and the assigned confidence rating to ensure that the code is appropriate for the medical entity.

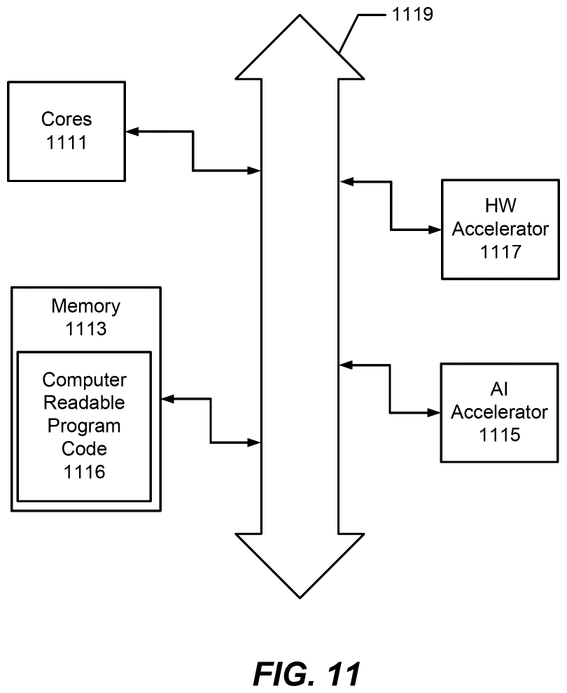

FIG. 11 is a block diagram of a data processing system that may be used to implement the coding suggestion server 140 of FIG. 1 and/or the medical entity extraction system 300, the medical ontology mapping system 400, and the validation system 500 of FIGS. 3-5 in accordance with some embodiments of the disclosure. As shown in FIG. 11, the data processing system may include at least one core 1111, a memory 1113, an artificial intelligence (AI) accelerator 1115 and a hardware (HW) accelerator 1117. The at least one core 1111, the memory 1113, the AI accelerator 1115, and the HW accelerator 1117 may communicate with each other through a bus 1119.

The at least one core 1111 may be configured to execute computer program instructions. For example, the at least one core 1111 may execute an operating system and/or applications represented by the computer readable program code 1116 stored in the memory 1113. In some embodiments, the at least one core 1111 may be configured to instruct the AI accelerator 1115 and/or the HW accelerator 1117 to perform operations by executing the instructions and obtain results of the operations from the AI accelerator 1115 and/or the HW accelerator 1117. In some embodiments, the at least one core 1111 may be an ASIP customized for specific purposes and support a dedicated instruction set.

The memory 1113 may have an arbitrary structure configured to store data. For example, the memory 1113 may include a volatile memory device, such as dynamic random-access memory (DRAM) and static RAM (SRAM), or include a non-volatile memory device, such as flash memory and resistive RAM (RRAM). The at least one core 1111, the AI accelerator 1115, and the HW accelerator 1117 may store data in the memory 1113 or read data from the memory 1113 through the bus 1119.

The AI accelerator 1115 may refer to hardware designed for AI applications. In some embodiments, the AI accelerator 1115 may include one or more machine learning models configured to provide an intelligent coding DSS. The AI accelerator 1115 may generate output data by processing input data provided from the at least one core 1115 and/or the HW accelerator 1117 and provide the output data to the at least one core 1111 and/or the HW accelerator 1117. In some embodiments, the AI accelerator 1115 may be programmable and be programmed by the at least one core 1111 and/or the HW accelerator 1117. The HW accelerator 1117 may include hardware designed to perform specific operations at high speed. The HW accelerator 1117 may be programmable and be programmed by the at least one core 1111.

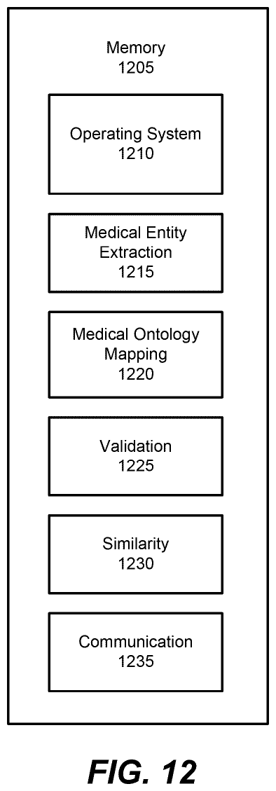

FIG. 12 illustrates a memory 1205 that may be used in embodiments of data processing systems, such as the coding suggestion server 140 of FIG. 1, the medical entity extraction system 300, the medical ontology mapping system 400, and the validation system 500, and the data processing system of FIG. 11, respectively, to provide an AI supplemented coding DSS for coding of medical entities extracted from clinical information. The memory 1205 is representative of the one or more memory devices containing the software and data used for facilitating operations of the coding suggestion server 140 and the DSS module 145 as described herein. The memory 1205 may include, but is not limited to, the following types of devices: cache, ROM, PROM, EPROM, EEPROM, flash, SRAM, and DRAM. As shown in FIG. 12, the memory 1205 may contain six or more categories of software and/or data: an operating system 1210, a medical entity extraction module 1215, a medical ontology mapping module 1220, a validation module 1225, a similarity module 1230, and a communication module 1235. In particular, the operating system 1210 may manage the data processing system's software and/or hardware resources and may coordinate execution of programs by the processor.

The medical entity extraction module 1215 may be configured to perform one or more of the operations described above with respect to the medical entity extraction system 300 of FIG. 3 and the flowcharts of FIGS. 6, 7, 9, and 10. The medical ontology mapping module 1220 may be configured to perform one or more of the operations described above with respect to the medical ontology mapping system 400 of FIG. 4 and the flowcharts of FIGS. 6, 7, 9, and 10. The validation module 1225 may be configured to perform one or more of the operations described above with respect to the validation system 500 and the flowcharts of FIGS. 6, 7, 9, and 10. The similarity module 1230 may be configured to perform one or more of the operations described above with respect to the medical ontology mapping system 400 of FIG. 4 and the flowcharts of FIGS. 6, 7, 9, and 10. The communication module 1235 may be configured to facilitate communication between the coding suggestion server 140 of FIG. 1 and/or the medical entity extraction system 300, the medical ontology mapping system 400, and the validation system 500 and the providers 110a, 110b, 110c, users 112a, 112b, medical entity knowledge base 170, and coding knowledge base of FIG. 1.

Although FIGS. 11 and 12 illustrate hardware/software architectures that may be used in data processing systems, such as the coding suggestion server 140 of FIG. 1, the medical entity extraction system 300, the medical ontology mapping system 400, and the validation system 500, and the data processing system of FIG. 11, respectively, in accordance with some embodiments of the disclosure, it will be understood that embodiments of the disclosure are not limited to such a configuration but is intended to encompass any configuration capable of carrying out operations described herein.

Computer program code for carrying out operations of data processing systems discussed above with respect to FIG. 1-12 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the intermediary server 130 of FIG. 1, the coding suggestion server 140 of FIG. 1, the medical entity extraction system 300, the medical ontology mapping system 400, the validation system 500, and the data processing system of FIG. 11 may each be implemented as a single processor system, a multi-processor system, a multi-core processor system, or even a network of stand-alone computer systems, in accordance with various embodiments of the disclosure. Each of these processor/computer systems may be referred to as a “processor” or “data processing system.” The functionality provided by the intermediary server 130 and the coding suggestion server 140 may be merged into a single server or maintained as separate servers in accordance with different embodiments of the disclosure.

The data processing apparatus described herein with respect to FIGS. 1-12 may be used to facilitate providing an AI supplemented coding DSS according to some embodiments of the disclosure described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1205 when coupled to a processor includes computer readable program code that, when executed by the processor, causes the processor to perform operations including one or more of the operations described herein with respect to FIGS. 1-12.

Some embodiments of the disclosure may provide an intelligent coding DSS that can, for example, assist medical coders and billers in coding medical entities during the claim generation workflow. The medical entity extraction model 210 may be an NER model that is built on a pre-trained deep learning model and fine-tuned to a custom dataset to allow medical entities to be extracted or highlighted with relatively high accuracy. A medical ontology mapping model 215 may identify relevant codes from among, for example, ICD-10, CPT, and HCPCS coding standards through a similarity analysis of embedding vectors of the extracted medical entities and codes. A validation model 220 may categorize the ranked codes that are candidates for each of the extracted medical entities with confidence ratings that reflect the varying degrees of confidence that candidate medical codes are correlated. Advantageously, the intelligent coding DSS may make use of AI models and medical knowledge repositories to improve both the efficiency and accuracy of the medical coding process.

Some embodiments of the disclosure may provide an intelligent decision support system as set forth by the following examples: Example 1: a computer-implemented method comprises: receiving, by one or more processors, a record containing clinical information associated with a patient; applying, by the one or more processors and to the record, a set of machine-learned models including: a medical entity extraction model that is configured to extract medical entities from the clinical information; a medical ontology mapping model that is configured to associate one or more candidate medical codes with each of the medical entities based on vector embedding similarities and to rank each of the one or more candidate medical codes based on their similarities, respectively, with the associated medical entity; and a validation model that is configured to categorize the association between one or more pairs of the one or more candidate medical codes into one or more confidence ratings, respectively; generating, by the one or more processors, a suggested medical code for each of the medical entities based on the rank of the one or more candidate medical codes associated therewith and the one or more confidence ratings of the one or more candidate medical codes, respectively; and causing, by the one or more processors and via a graphical user interface (GUI), display of the suggested medical code for each of the medical entities and, for each of the suggested medical codes, a corresponding visual indicator that is representative of the confidence rating that is associated with the respective suggested medical code.

Example 2: the computer-implemented method of Example 1, wherein the medical entity extraction model comprises a bidirectional encoder representations from transformers (BERT) model that is trained using annotated historic patient clinical information records.

Example 3: the computer-implemented method of any of Examples 1 and 2, wherein the method further comprises: generating, using the medical entity extraction model and the one or more processors, a set of candidate medical entities from the clinical information; generating, using the medical entity extraction model and the one or more processors, medical entity confidence scores for the candidate medical entities, respectively, using Gibbs sampling to perform a statistical inference based on the candidate medical entities and a medical entity knowledge base that contains quantified measures of relationship strength between pairs of reference medical entities; and extracting, by the one or more processors, the medical entities from the clinical information based on the confidence scores for the candidate medical entities.

Example 4: the computer-implemented method of any of Examples 1-3, wherein the method further comprises: categorizing, using the medical entity extraction model and the one or more processors, each of the extracted medical entities into one of a set of medical entity categories.

Example 5: the computer-implemented method of Example 4, wherein the set of medical entity categories comprises a disease category, a medication category, a dosage category, a form category, a route category, a procedure category, a lab category, and a symptom category.

Example 6: the computer-implemented method of any of Examples 1 - 5, wherein the method further comprises: generating, using the medical ontology mapping model and the one or more processors, a medical entity vector embedding for each of the medical entities; generating, using the medical ontology mapping model and the one or more processors, a reference code vector embedding for each of a set of reference medical codes; and determining, using the medical ontology mapping model and the one or more processors, the vector embedding similarities between ones of the medical entity vector embeddings and ones of the set of reference medical code vector embeddings.

Example 7: the computer-implemented method of Example 6, wherein determining the vector embedding similarities comprises: determining, using the medical ontology mapping model and the one or more processors, a set of logits corresponding to a set of inner products between the ones of the medical entity vector embeddings and the ones of the set of reference medical code vector embeddings.

Example 8: the computer-implemented method of Example 7, wherein determining the vector embedding similarities further comprises: applying, using the medical ontology mapping model and the one or more processors, a sigmoid function to each of the set of logits to generate a set of similarity probabilities.

Example 9: the computer-implemented method of Example 8, wherein: determining, using the medical ontology mapping model and the one or more processors, whether each of the set of similarity probabilities satisfies a similarity threshold to generate a set of similarity comparison results; associating, using the medical ontology mapping model and the one or more processors, the one or more candidate medical codes with each of the medical entities based on the similarity comparison results; and ranking, using the medical ontology mapping model and the one or more processors, each of the one or more candidate medical codes associated with each of the medical entities based on the similarity probabilities.

Example 10: the computer-implemented method of any of Claims 1-9, wherein the method further comprises: generating, using the validation model and the one or more processors, first confidence scores for reference medical code pairs based on conditional probabilities for the pairs, respectively; performing, using the validation model and the one or more processors, mean shift unsupervised clustering on the pairs to generate clusters; determining, using the validation model and the one or more processors, mean conditional probabilities for the clusters; generating, using the validation model and the one or more processors, second confidence scores for the reference medical code pairs based on the first confidence scores and the mean conditional probabilities for the clusters; and categorizing, for each of the set of medical entities and using the validation model and the one or more processors, the association between and one or more pairs of the one or more candidate medical codes into the one or more confidence ratings based on the second confidence scores, respectively.

Example 11: the computer-implemented method of Example 10, wherein the one or more confidence ratings reflect varying degrees of confidence that a respective one of the reference medical entities and a respective one the reference medical codes are correlated.

Example 12: the computer-implemented method of any of Examples 1-11, wherein the suggested medical code is an International Classification of Diseases (ICD) code, a Current Procedural Terminology (CPT) code, or a Healthcare Common Procedure Coding System (HCPCS) code.

Example 13: a system comprises one or more processors; and one or more memories storing process-executable instructions that, when executed by the one or more processors, cause the one or more processors to perform operations comprising: receiving a record containing clinical information associated with a patient; applying, to the record, a set of machine-learned models including: a medical entity extraction model that is configured to extract medical entities from the clinical information; a medical ontology mapping model that is configured to associate one or more candidate medical codes with each of the medical entities based on vector embedding similarities and to rank each of the one or more candidate medical codes based on their similarities, respectively, with the associated medical entity; and a validation model that is configured to categorize the association between one or more pairs of the one or more candidate medical codes into one or more confidence ratings, respectively; generating a suggested medical code for each of the medical entities based on the rank of the one or more candidate medical codes associated therewith and the one or more confidence ratings of the one or more candidate medical codes, respectively; and causing, via a graphical user interface (GUI), display of the suggested medical code for each of the medical entities and, for each of the suggested medical codes, a corresponding visual indicator that is representative of the confidence rating that is associated with the respective suggested medical code.

Example 14: the system of Example 13, wherein the medical entity extraction model comprises a bidirectional encoder representations from transformers (BERT) model that is trained using annotated historic patient clinical information records.

Example 15: the system of any of Examples 12 and 13, wherein the operations further comprise: generating, using the medical entity extraction model, a set of candidate medical entities from the clinical information; generating, using the medical entity extraction model, medical entity confidence scores for the candidate medical entities, respectively, using Gibbs sampling to perform a statistical inference based on the candidate medical entities and a medical entity knowledge base that contains quantified measures of relationship strength between pairs of reference medical entities; and extracting the medical entities from the clinical information based on the confidence scores for the candidate medical entities.

Example 16: the system of any of Examples 12-15, wherein the operations further comprise: categorizing, using the medical entity extraction model, each of the extracted medical entities into one of a set of medical entity categories.

Example 17: the system of any of Examples 12-16, wherein the operations further comprise: generating, using the medical ontology mapping model, a medical entity vector embedding for each of the medical entities; generating, using the medical ontology mapping model, a reference code vector embedding for each of a set of reference medical codes; determining, using the medical ontology mapping model, the vector embedding similarities between ones of the medical entity vector embeddings and ones of the set of reference medical code vector embeddings.

Example 18, the system of any of Examples 12-17, the operations further comprise: generating, using the validation model, first confidence scores for reference medical code pairs based on conditional probabilities for the pairs, respectively; performing, using the validation model, mean shift unsupervised clustering on the pairs to generate clusters; determining, using the validation model, mean conditional probabilities for the clusters; generating, using the validation model, second confidence scores for the reference medical code pairs based on the first confidence scores and the mean conditional probabilities for the clusters; and categorizing, for each of the set of medical entities and using the validation model, the association between and one or more pairs of the one or more candidate medical codes into the one or more confidence ratings based on the second confidence scores, respectively.

Example 19: the system of Example 18, wherein the one or more confidence ratings reflect varying degrees of confidence that respective ones of the one or more pairs of the one or more candidate medical codes are correlated.

Example 20: One or more non-transitory computer-readable media storing processor-executable instructions that, when executed by one or more processors, cause the one or more processors to perform operations comprising: receiving a record containing clinical information associated with a patient; applying, to the record, a set of machine-learned models including: a medical entity extraction model that is configured to extract medical entities from the clinical information; a medical ontology mapping model that is configured to associate one or more candidate medical codes with each of the medical entities based on vector embedding similarities and to rank each of the one or more candidate medical codes based on their similarities, respectively, with the associated medical entity; and a validation model that is configured to categorize the association between one or more pairs of the one or more candidate medical codes into one or more confidence ratings, respectively; generating a suggested medical code for each of the medical entities based on the rank of the one or more candidate medical codes associated therewith and the one or more confidence ratings of the one or more candidate medical codes, respectively; and causing, via a graphical user interface (GUI), display of the suggested medical code for each of the medical entities and, for each of the suggested medical codes, a corresponding visual indicator that is representative of the confidence rating that is associated with the respective suggested medical code.

### Conclusion

Throughout this specification, components, operations, or structures described as a single instance may be implemented as multiple instances. Although individual operations of one or more methods (or processes, techniques, routines, etc.) are illustrated and described as separate operations, two or more of the individual operations may be performed concurrently or otherwise in parallel, and nothing requires that the operations be performed in the order illustrated. Structures and functionality (e.g., operations, steps, blocks) presented as separate components in example configurations may be implemented as a combined structure, functionality, or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, operations, blocks, or instructions. These may constitute and/or be implemented by software (e.g., code embodied on a non-transitory, machine-readable medium), hardware, or a combination thereof. In hardware, the routines, etc., may represent tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware component that operates to perform certain operations as described herein.

In various embodiments, a hardware component may be implemented mechanically or electronically. For example, a hardware component may comprise dedicated circuitry or logic that is permanently configured (e.g., as a special-purpose processor, such as a field programmable gate array (FPGA) or an application-specific integrated circuit (ASIC)) to perform certain operations. A hardware component may also or instead comprise programmable logic or circuitry (e.g., as encompassed within one or more general-purpose processors and/or other programmable processor(s)) that is temporarily configured by software to perform certain operations.

Accordingly, the term “hardware component” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where the hardware components include a general-purpose processor configured using software, the general-purpose processor may be configured as respective different hardware components at different times. Software may accordingly configure a processor, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple of such hardware components exist contemporaneously, communications may be achieved through signal transmission (e.g., over appropriate circuits and buses) that connect the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information).

As noted above, the various operations of example methods (or processes, techniques, routines, etc.) described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions. The components referred to herein may, in some example embodiments, comprise processor-implemented components.

Moreover, each operation of processes illustrated as logical flow graphs may represent a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

The terms “coupled” and “connected,” along with their derivatives, may be used. In particular embodiments, “connected” may be used to indicate that two or more elements are in direct physical or electrical contact with each other, although the context in the description may dictate otherwise when it is apparent that two or more elements are not in direct physical or electrical contact. “Coupled” may mean that two or more elements are in direct physical or electrical contact. However, “coupled” may also mean that two or more elements are not in direct contact with each other, yet still co-operate, transmit between, or interact with each other.

An algorithm may be considered to be a self-consistent sequence of acts or operations leading to a desired result. These include physical manipulations of physical quantities. Usually, though not necessarily, these quantities take the form of electrical, magnetic, or optical signals capable of being stored, transferred, combined, compared, and otherwise manipulated. These signals are commonly referred to as bits, values, elements, symbols, characters, terms, numbers, flags, or the like. It should be understood, however, that all of these and similar terms are to be associated with the appropriate physical quantities and are merely convenient labels applied to these quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “some embodiments,” “one embodiment,” “an embodiment,” “in some examples,” or variations thereof means that a particular element, feature, structure, characteristic, operation, or the like described in connection with the embodiment is included in at least one embodiment, but not every embodiment necessarily includes the particular element, feature, structure, characteristic, operation, or the like. Different instances of such a reference in various places in the specification do not necessarily all refer to the same embodiment, although they may in some cases. Moreover, different instances of such a reference may describe elements, features, structures, characteristics, operations, or the like be combined in any manner as an embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless the context of use clearly indicates otherwise, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “set” is intended to mean a collection of elements and can be a null set (i.e., a set containing zero elements) or may comprise one, two, or more elements. A “subset” is intended to mean a collection of elements that are all elements of a set, but that does not include other elements of the set. A first subset of a set may comprise zero, one, or more elements that are also elements of a second subset of the set. The first subset may be said to be a subset of the second subset if all the elements of the first subset are elements of the second subset, while also being a subset of the set. However, if all the elements of the second subset are also elements of the first subset (in addition to all the elements of the first subset being elements of the second subset), the first subset and the second subset are a single subset/not distinct.

For the purposes of the present disclosure, the term “a” or “an” entity refers to one or more of that entity. As such, the terms “a” or “an”, “one or more”, and “at least one” can be used interchangeably herein unless explicitly contradicted by the specification using the word “only one” or similar. For example, “a first element” may functionally be interpreted as “a first one or more elements” or a “first at least one element.” Unless otherwise apparent from the context of use, reference in the present disclosure to a same set of “one or more processors” (or a same “plurality of processors,” etc.) performing multiple operations can encompass implementations in which performance of the operations is divided among the processor(s) in any suitable way. For example, “generating, by one or more processors, X; and generating, by the one or more processors, Y” can encompass: (1) implementations in which a first subset of the processors (e.g., in a first computing device) generates X and an entirely distinct, second subset of the processors (e.g., in a different, second computing device) independently generates Y; (2) implementations in which one or more or all of the processor(s) (e.g., one or multiple processors in the same device, or multiple processors distributed among multiple devices) contribute to the generation of X and/or Y; and (3) other variations. This may similarly be applied to any other component or feature similarly recited (e.g., as “a component”, “a feature”, “one or more components”, “one or more features”, “a plurality of components”, “a plurality of features”). Moreover, the performance of certain of the operations may be distributed among the one or more components, not only residing within a single machine, but deployed across a number of machines. The set of components may be located in a single geographic location (e.g., within a home environment, an office environment, a cloud environment). In other example embodiments, the set of components may be distributed across two or more geographic locations. Further, “a machine-learned model”, equivalent terms (e.g., “machine learning model,” “machine-learning model,” “machine-learned component”, “artificial intelligence”, “artificial intelligence component”), or species thereof (e.g., “a large language model”, “a neural network”) may include a single machine-learned model or multiple machine-learned models, such as a pipeline comprising two or more machine-learned models arranged in series and/or parallel, an agentic framework of machine-learned models, or the like.

An “artificial intelligence” or “artificial intelligence component” may comprise a machine-learned model. A machine-learned model may comprise a hardware and/or software architecture having structural hyperparameters defining the model's architecture and/or one or more parameters (e.g., coefficient(s), weight(s), biase(s), activation function(s) and/or action function type(s) in examples where the activation function and/or function type is determined as part of training, clustering centroid(s)/medoid(s), partition(s), number of trees, tree depth, split parameters) determined as a result of training the machine-learned model based at least in part on training hyperparameters (e.g., for supervised, semi-supervised, and reinforcement learning models) and/or by iteratively operating the machine-learned model according to the training hyperparameters(e.g., for unsupervised machine-learned models).

In some examples, structural hyperparameter(s) may define component(s) of the model's architecture and/or their configuration/order, such as, for example, the configuration/order specifying which input(s) are provided to one component and which output(s) of that component are provided as input to other component(s) of the machine-learned model; a number, type, and/or configuration of component(s) per layer; a number of layers of the model; a number and/or type of input nodes in an input layer of the model; a number and/or type of nodes in a layer; a number and/or type of output nodes of an output layer of the model; component dimension (e.g., input size versus output size); a number of trees; a maximum tree depth; node split parameters; minimum number of samples in a leaf node of a tree; and/or the like. The component(s) of the model may comprise one or more activation functions and/or activation function type(s) (e.g., gated linear unit (GLU), such as a rectified linear unit (ReLU), leaky RELU, Gaussian error linear unit (GELU), Swish, hyperbolic tangent), one or more attention mechanism and/or attention mechanism types (e.g., self-attention, cross-attention), nodes and split indications and/or probabilities in a decision tree, and/or various other component(s) (e.g., adding and/or normalization layer, pooling layer, filter). Various combinations of any these components (as defined by the structural hyperparameter(s)) may result in different types of model architectures, such as a transformer-based machine-learned model (e.g., encoder-only model(s), encoder-decoder model(s), decoder-only models, generative pre-trained transformer(s) (GPT(s))), neural network(s), multi-layer perceptron(s), Kolmogorov-Arnold network(s), clustering algorithm(s), support vector machine(s), gradient boosting machine(s), and/or the like. The structural parameters and components a machine-learned model comprises may vary depending on the type of machine-learned model.

Training hyperparameter(s) may be used as part of training or otherwise determining the machine-learned model. In some examples, the training hyperparameter(s), in addition to the training data and/or input data, may affect determining the parameter(s) of the target machine-learned model. Using a different set of training hyperparameters to train two machine-learned models that have the same architecture (i.e., the same structural hyperparameters) and using the same training data may result in the parameters of the first machine-learned model differing from the parameters of the second machine-learned model. Despite having the same architecture and having been trained using the same training data, such machine-learned models may generate different outputs from each other, given the same input data. Accordingly, accuracy, precision, recall, and/or bias may vary between such machine-learned models.

In some examples, training hyperparameter(s) may include a train-test split ratio, activation function and/or activation function type (e.g., in examples like Kolmogorov-Arnold networks (KANs) where the activation function type is determined as part of training from an available set of activation functions and/or limits on the activation function parameters specified by the training hyperparameters), training stage(s) (e.g., using a first set of hyperparameters for a first epoch of training, a second set of hyperparameters for a second epoch of training), a batch size and/or number of batches of data in a training epoch, a number of epochs of training, the loss function used (e.g., L1, L2, Huber, Cauchy, cross entropy), the component(s) of the machine-learned model that are altered using the loss for a particular batch or during a particular epoch of training (e.g., some components may be “frozen,” meaning their parameters are not altered based on the loss), learning rate, learning rate optimization algorithm type (e.g., gradient descent, adaptive, stochastic) used to determine an alteration to one or more parameters of one or more components of the machine-learned model to reduce the loss determined by the loss function, learning rate scheduling, and/or the like.

In some examples, the structural hyperparameters and/or the training hyperparameters may be determined by a hyperparameter optimization algorithm or based on user input, such as a software component written by a user or generated by a machine-learned model. The machine-learned model may include any type of model configured, trained, and/or the like to generate a prediction output for a model input. In some examples, any of the logic, component(s), routines, and/or the like discussed herein may be implemented as a machine-learned model.

The machine-learned model may include one or more of any type of machine-learned model including one or more supervised, unsupervised, semi-supervised, and/or reinforcement learning models. Training a machine-learned model may comprise altering one or more parameters of the machine-learned model (e.g., using a loss optimization algorithm) to reduce a loss. Depending on whether the machine-learned model is supervised, semi-supervised, unsupervised, etc. this loss may be determined based at least in part on a difference between an output generated by the model and ground truth data (e.g., a label, an indication of an outcome that resulted from a system using the output), a cost function, a fit of the parameter(s) to a set of data, a fit of an output to a set of data, and/or the like. In some examples, determining an output by a machine-learned model may comprise executing a set of inference operations executed by the machine-learned model according to the target machine-learned model's parameter(s) and structural hyperparameter(s) and using/operating on a set of input data.

Moreover, any discussion of receiving data associated with an individual that may be protected, confidential, or otherwise sensitive information, is understood to have been preceded by transmitting a notice of use of the data to a computing device, account, or other identifier (collectively, “identifier”) associated with the individual, receiving an indication of authorization to use the data from the identifier, and/or providing a mechanism by which a user may cause use of the data to cease or a copy of the data to be provided to the user.

Upon reading this disclosure, those of skill in the art will appreciate still additional alternative structural and functional designs through the principles disclosed herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The patent claims at the end of this patent application are not intended to be construed under 35 U.S. C. § 112(f) unless traditional means-plus-function language is expressly recited, such as “means for” or “step for” language being explicitly recited in the claim(s).

The description of the embodiments of the present disclosure has been presented for purposes of illustration and description, but is not intended to be exhaustive or limited to the embodiments in the form disclosed. Many modifications and variations will be apparent to those of ordinary skill in the art without departing from the scope and spirit of the disclosed embodiments. The aspects of the disclosure herein were chosen and described to best explain the principles of the embodiments and the practical application, and to enable others of ordinary skill in the art to understand the embodiments with various modifications as are suited to the particular use contemplated.

What is claimed is:

1. A computer-implemented method, comprising:
receiving, by one or more processors, a record containing clinical information associated with a patient;
applying, by the one or more processors and to the record, a set of machine-learned models including:
a medical entity extraction model that is configured to extract medical entities from the clinical information;
a medical ontology mapping model that is configured to associate one or more candidate medical codes with each of the medical entities based on vector embedding similarities and to rank each of the one or more candidate medical codes based on their similarities, respectively, with the associated medical entity; and
a validation model that is configured to categorize the association between one or more pairs of the one or more candidate medical codes into one or more confidence ratings, respectively;
generating, by the one or more processors, a suggested medical code for each of the medical entities based on the rank of the one or more candidate medical codes associated therewith and the one or more confidence ratings of the one or more candidate medical codes, respectively; and
causing, by the one or more processors and via a graphical user interface (GUI), display of the suggested medical code for each of the medical entities and, for each of the suggested medical codes, a corresponding visual indicator that is representative of the confidence rating that is associated with the respective suggested medical code.

2. The computer-implemented method of claim 1, wherein the medical entity extraction model comprises a bidirectional encoder representations from transformers (BERT) model that is trained using annotated historic patient clinical information records.

3. The computer-implemented method of claim 2, further comprising:
generating, using the medical entity extraction model and the one or more processors, a set of candidate medical entities from the clinical information;
generating, using the medical entity extraction model and the one or more processors, medical entity confidence scores for the candidate medical entities, respectively, using Gibbs sampling to perform a statistical inference based on the candidate medical entities and a medical entity knowledge base that contains quantified measures of relationship strength between pairs of reference medical entities; and
extracting, by the one or more processors, the medical entities from the clinical information based on the confidence scores for the candidate medical entities.

4. The computer-implemented method of claim 3, further comprising:
categorizing, using the medical entity extraction model and the one or more processors, each of the extracted medical entities into one of a set of medical entity categories.

5. The computer-implemented method of claim 3, wherein the set of medical entity categories comprises a disease category, a medication category, a dosage category, a form category, a route category, a procedure category, a lab category, and a symptom category.

6. The computer-implemented method of claim 1, further comprising:
generating, using the medical ontology mapping model and the one or more processors, a medical entity vector embedding for each of the medical entities;
generating, using the medical ontology mapping model and the one or more processors, a reference code vector embedding for each of a set of reference medical codes; and
determining, using the medical ontology mapping model and the one or more processors, the vector embedding similarities between ones of the medical entity vector embeddings and ones of the set of reference medical code vector embeddings.

7. The computer-implemented method of claim 6, wherein determining the vector embedding similarities comprises:
determining, using the medical ontology mapping model and the one or more processors, a set of logits corresponding to a set of inner products between the ones of the medical entity vector embeddings and the ones of the set of reference medical code vector embeddings.

8. The computer-implemented method of claim 7, wherein determining the vector embedding similarities further comprises:
applying, using the medical ontology mapping model and the one or more processors, a sigmoid function to each of the set of logits to generate a set of similarity probabilities.

9. The computer-implemented method of claim 8, further comprising:
determining, using the medical ontology mapping model and the one or more processors, whether each of the set of similarity probabilities satisfies a similarity threshold to generate a set of similarity comparison results;
associating, using the medical ontology mapping model and the one or more processors, the one or more candidate medical codes with each of the medical entities based on the similarity comparison results; and
ranking, using the medical ontology mapping model and the one or more processors, each of the one or more candidate medical codes associated with each of the medical entities based on the similarity probabilities.

10. The computer-implemented method of claim 1, further comprising:
generating, using the validation model and the one or more processors, first confidence scores for reference medical code pairs based on conditional probabilities for the pairs, respectively;
performing, using the validation model and the one or more processors, mean shift unsupervised clustering on the pairs to generate clusters;
determining, using the validation model and the one or more processors, mean conditional probabilities for the clusters;
generating, using the validation model and the one or more processors, second confidence scores for the reference medical code pairs based on the first confidence scores and the mean conditional probabilities for the clusters; and
categorizing, for each of the set of medical entities and using the validation model and the one or more processors, the association between one or more pairs of the one or more candidate medical codes into the one or more confidence ratings based on the second confidence scores, respectively.

11. The computer-implemented method of claim 10, wherein the one or more confidence ratings reflect varying degrees of confidence that respective ones of the one or more pairs of the one or more candidate medical codes are correlated.

12. The computer-implemented method of claim 1, wherein the suggested medical code is an International Classification of Diseases (ICD) code, a Current Procedural Terminology (CPT) code, or a Healthcare Common Procedure Coding System (HCPCS) code.

13. A system, comprising:
one or more processors; and
one or more memories storing process-executable instructions that, when executed by the one or more processors, cause the one or more processors to perform operations comprising:
receiving a record containing clinical information associated with a patient;
applying to the record, a set of machine-learned models including:
a medical entity extraction model that is configured to extract medical entities from the clinical information;
a medical ontology mapping model that is configured to associate one or more candidate medical codes with each of the medical entities based on vector embedding similarities and to rank each of the one or more candidate medical codes based on their similarities, respectively, with the associated medical entity; and
a validation model that is configured to categorize an association between one or more pairs of the one or more candidate medical codes into one or more confidence ratings, respectively;
generating a suggested medical code for each of the medical entities based on the rank of the one or more candidate medical codes associated therewith and the one or more confidence ratings of the one or more candidate medical codes, respectively; and
causing, via a graphical user interface (GUI), display of the suggested medical code for each of the medical entities and, for each of the suggested medical codes, a corresponding visual indicator that is representative of the confidence rating that is associated with the respective suggested medical code.

14. The system of claim 13, wherein the medical entity extraction model comprises a bidirectional encoder representations from transformers (BERT) model that is trained using annotated historic patient clinical information records.

15. The system of claim 14, wherein the operations further comprise:
generating, using the medical entity extraction model, a set of candidate medical entities from the clinical information;
generating, using the medical entity extraction model, medical entity confidence scores for the candidate medical entities, respectively, using Gibbs sampling to perform a statistical inference based on the candidate medical entities and a medical entity knowledge base that contains quantified measures of relationship strength between pairs of reference medical entities; and
extracting the medical entities from the clinical information based on the confidence scores for the candidate medical entities.

16. The system of claim 15, wherein the operations further comprise:
categorizing, using the medical entity extraction model, each of the extracted medical entities into one of a set of medical entity categories.

17. The system of claim 13, wherein the operations further comprise:
generating, using the medical ontology mapping model, a medical entity vector embedding for each of the medical entities;
generating, using the medical ontology mapping model, a reference code vector embedding for each of a set of reference medical codes; and
determining, using the medical ontology mapping model, the vector embedding similarities between ones of the medical entity vector embeddings and ones of the set of reference medical code vector embeddings.

18. The system of claim 13, wherein the operations further comprise:
generating, using the validation model, first confidence scores for reference medical code pairs based on conditional probabilities for the pairs, respectively;
performing, using the validation model, mean shift unsupervised clustering on the pairs to generate clusters;
determining, using the validation model, mean conditional probabilities for the clusters;
generating, using the validation model, second confidence scores for the reference medical code pairs based on the first confidence scores and the mean conditional probabilities for the clusters; and
categorizing, for each of the set of medical entities and using the validation model, the association between and one or more pairs of the one or more candidate medical codes into the one or more confidence ratings based on the second confidence scores, respectively.

19. The system of claim 18, wherein the one or more confidence ratings reflect varying degrees of confidence that respective ones of the one or more pairs of the one or more candidate medical codes are correlated.

20. One or more non-transitory computer-readable media storing processor-executable instructions that, when executed by one or more processors, cause the one or more processors to perform operations comprising:
receiving a record containing clinical information associated with a patient;
applying, to the record, a set of machine-learned models including:
a medical entity extraction model that is configured to extract medical entities from the clinical information;
a medical ontology mapping model that is configured to associate one or more candidate medical codes with each of the medical entities based on vector embedding similarities and to rank each of the one or more candidate medical codes based on their similarities, respectively, with the associated medical entity; and
a validation model that is configured to categorize, an association between one or more pairs of the one or more candidate medical codes into one or more confidence ratings, respectively;
generating a suggested medical code for each of the medical entities based on the rank of the one or more candidate medical codes associated therewith and the one or more confidence ratings of the one or more candidate medical codes, respectively; and
causing, via a graphical user interface (GUI), display of the suggested medical code for each of the medical entities and, for each of the suggested medical codes, a corresponding visual indicator that is representative of the confidence rating that is associated with the respective suggested medical code.
